(19) United States
(12) Patent Application Publication
(10) Pub. No.: US 2018/0292380 A1
(43) Pub. Date: Oct. 11, 2018

(54) MULTI-LIQUID QUALITY CALIBRATION SINGLE-USE CARTRIDGE

(71) Applicant: LifeHealth, LLC, Roseville, MN (US)

(72) Inventors: James D. Kurkowski, Roseville, MN (US); Nancy Copeland Ring, New Brighton, MN (US); Gregory Scott Buell, Plymouth, MN (US)

(21) Appl. No.: 15/481,698

(22) Filed: Apr. 7, 2017

(51) Int. Cl. G01N 33/487; G01N 33/48

(52) U.S. Cl. CPC G01N 33/48771; G01N 33/48

(57) ABSTRACT

A method of evaluating a POC system that includes loading a first LQC fluid onto the testing chamber and measuring the first LQC fluid with the sensor to obtain a first actual measurement, where the first LQC fluid can comprise a first known concentration of an analyte. The method also includes loading a second LQC fluid, having a second known concentration of the at least one analyte, into the testing chamber and measuring the second LQC fluid with the sensor to obtain a second actual measurement. The known concentrations are evaluated against the actual measurements to determine difference values. The values are compared against expected LQC values to determine if the cartridge, the cartridge manufacturing lot, the POC system, or the user proficiency are outside quality control expectations.

### TECHNICAL FIELD

This document pertains generally, but not by way of limitation, to single use test cartridges for calibrating and evaluating point of care testing devices.

### BACKGROUND

Point of care (“POC”) testing devices are used to evaluate collected biological samples immediately following or soon after collection of the samples. POC testing devices can receive single use cartridges with integrated sensors, wherein biological samples are loaded onto the removable cartridge for evaluation.

Sensor cartridges that are single use devices are typically produced in large lots preventing individual testing before sale. Instead, each lot is spot checked for quality at the manufacturer. Customers also often check the lot quality at the point of use to determine if shipping and storage conditions were met and that the lot is continuing to perform as expected. The point of use check is performed by loading the cartridge with a liquid quality control (“LQC”) containing a reagent formulated to provide known results. By comparing the expected results against the actual results, the operation of the cartridge or the proficiency of the users can be evaluated.

Government regulation or hospital procedures often require that the POC testing systems are reevaluated at regular intervals (e.g. daily or at the beginning of each shift). A liquid quality control (“LQC”) fluid formulated to provide a known sensor measurement for one or more analyte is fed into the single use cartridge in place of a biological sample. Typically, LQC testing involves evaluating at least three different LQC fluids—a “low” LQC fluid corresponding to a low range boundary of at least one analyte, a “high” LQC fluid corresponding to a high range boundary of the analyte, and a “mid” LQC fluid corresponding to a value within the range of the analyte. The testing of the LQC fluids is time consuming as each LQC fluid measurements requires a full cartridge test sequence to obtain results. In addition, each LQC test cycle consumes a single use sensor cartridge. Each consumed sensor cartridge could otherwise be used to test for a biological sample.

### OVERVIEW

The present inventors have recognized, among other things, that a problem to be solved can include time consuming evaluation of POC systems and the consumption of the single-use cartridges to evaluate the POC system. In an example, the present subject matter can provide a solution to this problem, such as by a single-use cartridge defining a flow path extending through a testing chamber within which a sensor is positioned. The sensor can be configured to measure at least one analyte in a fluid received within the testing chamber. The flow path configuration of the testing chamber allows multiple aliquots of calibration fluids, LQC fluids, and combinations thereof to be passed through one single-use cartridge for evaluation with the sensor. In this configuration, a single cartridge can be used to perform the regular calibration and quality control evaluations of the sensor and system mandated by health procedures or government regulations.

In an example, a method of evaluating a POC system can include loading a first LQC fluid onto the testing chamber and measuring the first LQC fluid with the sensor to obtain a first actual measurement. The first LQC fluid can comprise a first known concentration of the at least one analyte. The first known concentration can be evaluated against the first actual measurement to determine a first difference value. The method can include loading a second LQC fluid, having a second known concentration of the at least one analyte, into the testing chamber and measuring the second LQC fluid with the sensor to obtain a second actual measurement. The second known concentration can be evaluated against the second actual measurement to determine a second difference value. The first and second difference values are compared against expected LQC values to determine if the specific cartridge, the cartridge manufacturing lot, the POC system, or the user proficiency are within quality control expectations.

This overview is intended to provide an overview of subject matter of the present patent application. It is not intended to provide an exclusive or exhaustive explanation of the present subject matter. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic view of a POC system according to an example of the present disclosure.

FIG. 2A is a schematic view of a method of taking multiple LQC measurements on a single use cartridge according to an example of the present disclosure.

FIG. 2B is a schematic view of a method of taking multiple LQC measurements on a single use cartridge according to an example of the present disclosure.

FIG. 3 is a representative sensor response chart of multiple LQC measurements taken on a single use cartridge according to an example of the present disclosure.

### DETAILED DESCRIPTION

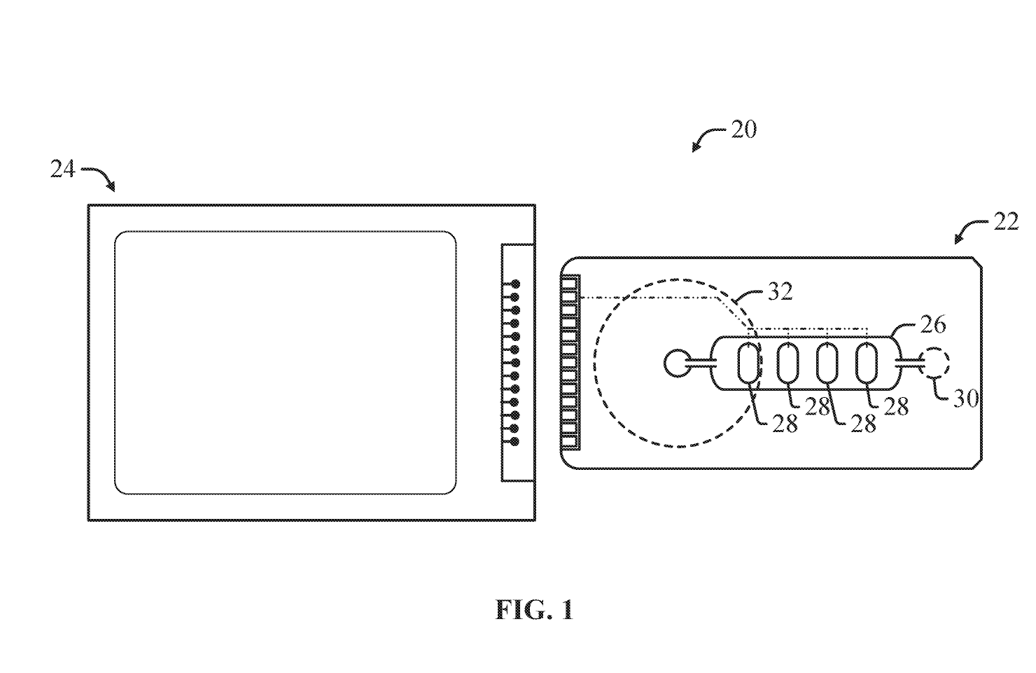

As depicted in FIG. 1, a point-of-care (“POC”) system 20, according to an example of the present disclosure, can include a single-use cartridge 22 configured to interface with a POC system 24. The single-use cartridge 22 can receive at least one fluid and measure the concentration of at least one analyte within the fluid. The sensor information can be communicated from the single-use cartridge 22 to the POC system 24 for evaluation, processing, and display of the sensor information. In this configuration, the single-use cartridge 22 can include at least one interface contact configured to interface with a corresponding contact of the POC system 24. As defined in the present application, a “single-use” cartridge 22 can comprise cartridges 22 configured to receive multiple aliquots of fluid to perform a series of tests on a single cartridge 22. For example, a single use cartridge 22 can be configured to receive an aliquot of a calibration fluid for calibrating a sensor of the cartridge 22 and at least one aliquot of an LQC fluid for evaluating the sensor. In certain examples, multiple aliquots of LQC fluid can be loaded onto the single cartridge 22 for evaluating multiple parameters of the sensor. In certain examples, an aliquot of a biological sample can be loaded onto the single use cartridge 22 in addition to at least the calibration fluid with or without the LQC fluids.

As depicted in FIG. 1, the single-use cartridge 22 can define a flow path extending through a testing chamber 26 and including at least one sensor 28 positioned within the testing chamber 26. Each sensor 28 can be configured to measure at least one analyte contained in a fluid received within the testing chamber 26. A feed port 30 can be fluidically connected to the flow path upstream of the testing chamber 26. In operation, fluid can be fed into the flow path through the feed port 30 and into the testing chamber 26. A waste chamber 32 can be fluidly connected to the flow path downstream of the testing chamber 26 for receiving fluid displaced from the testing chamber 26. In operation, loading fluid into the testing chamber 26 can displace fluid already in the testing chamber 26 and forcing the displaced fluid into the waste chamber 32.

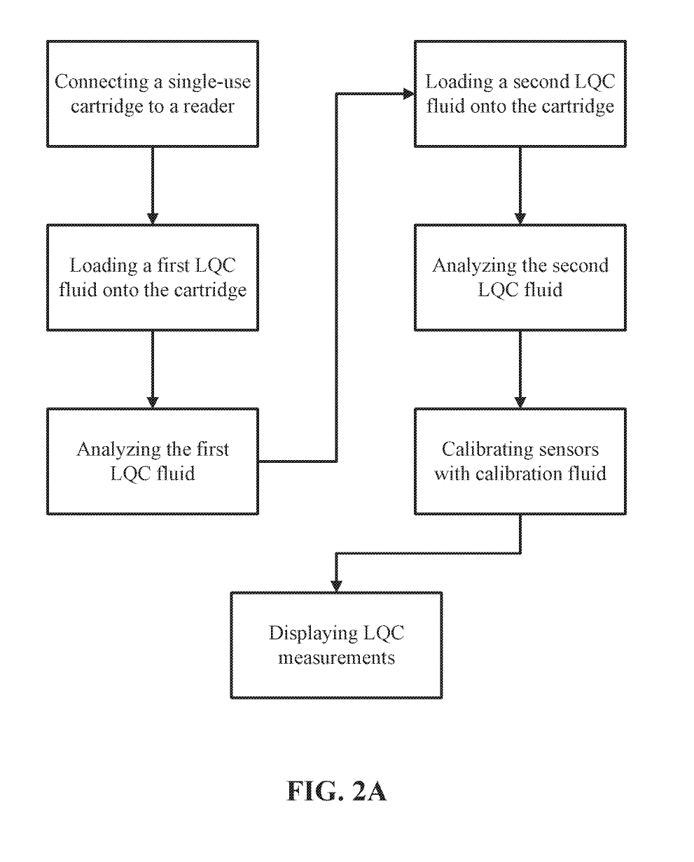

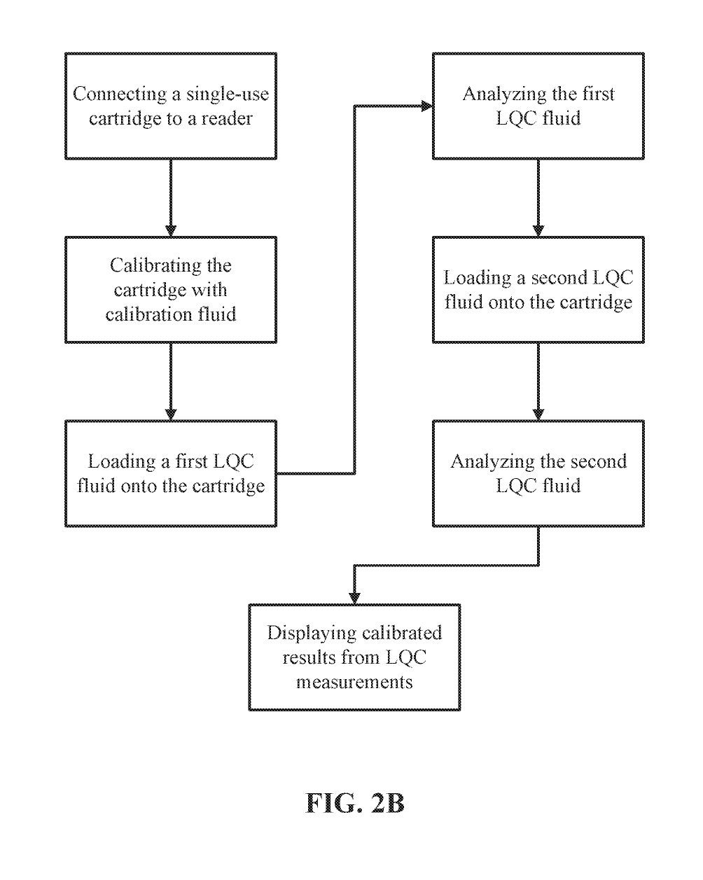

As depicted in FIGS. 2A-B, a method for evaluation of the POC system 20 can include multiple calibrations and/or quality control evaluations of the sensor 28 with one single-use cartridge 22. In particular, the flow path configuration of the testing chamber 26 allows multiple aliquots of calibration fluids, LQC fluids, and combinations thereof to be passed through one single-use cartridge 22. In this configuration, fewer cartridges are required to perform the regular calibration and quality control evaluations of the sensor 28 mandated by health procedures or government regulations.

In an example, as depicted in FIG. 2A, the method can comprise loading the testing chamber 26 with a first LQC fluid. The first LQC fluid can have a first known concentration of the at least one analyte. The first known concentration can correspond to a lower bound of an expected measurement range for the analyte in a biological sample; an upper bound of the expected measurement range; or a midpoint within the expected measurement range. In an example, the first LQC fluid can include more than one analyte each with a first known concentration.

The method can comprise taking a measurement of the first LQC fluid with the sensor 28 of the single-use cartridge 22 to obtain an actual first measurement of the analyte in the first LQC fluid.

The method can comprise loading the testing chamber 26 with a second LQC fluid. The second LQC fluid can displace the first LQC fluid from the testing chamber 26 and into the waste chamber 32. The second LQC fluid can have a second known concentration of the at least one analyte. The second known concentration can correspond to a lower bound of an expected measurement range for the analyte in a biological sample; an upper bound of the expected measurement range; or a midpoint within the expected measurement range. The second LQC fluid can have a second known concentration that differs from the first known concentration of the first LQC fluid such that the second known concentration corresponds to a different portion of the expected measurement range. In an example, the LQC fluids can include more than one analyte each with a known concentration.

The method can comprise taking a measurement of the second LQC fluid with the sensor 28 of the single-use cartridge 22 to obtain an actual second measurement of the analyte in the second LQC fluid.

As illustrated in FIG. 2B, the method can comprise loading the testing chamber 26 with a calibration fluid. The calibration fluid can be pre-loaded into the testing chamber 26 or loaded prior to the first LQC fluid such that the first LQC fluid displaces the calibration fluid from the testing chamber 26 and into the waste chamber 32. Alternatively, the calibration fluid can be loaded into the testing chamber 26 after the second LQC fluid to displace the second LQC fluid from the testing chamber 26 and into the waste chamber 32. The calibration fluid can be formulated to provide a known calibration value for at least one analyte of the biological sample on the particular sensor 28 of the single-use cartridge 22. The calibration fluid is selected and formulated to account for the particular manufacturing variances of the sensor 28 (or manufacturing lot of sensors 28); expected shelf life of the sensor 28; type of biological fluid to be evaluated; and other factors affecting performance of the specific sensor 28.

A calibration measurement can be taken of the calibration fluid to obtain an actual calibration value for the at least one analyte. The actual calibration value can be compared against the reference calibration value to determine an offset value corresponding the difference between the actual calibration value and the known reference calibration value. The offset value can be applied to the actual first and second measurements of the first and second LQC fluids, respectively.

The first and second actual measurements can be compared with the first and second known concentrations to determine a first difference value and a second difference values between the actual measurements and the known concentrations. The POC system can be configured to display the first and second measurements and difference values for record taking purposes. The POC system can be configured to provide a notification on the display of the POC system if the first or second difference values exceed a predetermined threshold.

In an example, as depicted in FIG. 2B, the method can comprise loading the testing chamber 26 with a calibration fluid. The calibration fluid can be formulated to provide a known calibration value for at least one analyte of the biological sample on the particular sensor 28 of the single-use cartridge 22. The calibration fluid is selected and formulated to account for the particular manufacturing variances of the sensor 28 (or manufacturing lot of sensors 28); expected shelf life of the sensor 28; type of biological fluid to be evaluated; and other factors affecting performance of the specific sensor 28. In an example, the testing chamber 26 of the single-use cartridge 22 can be pre-loaded with the calibration fluid for protecting the sensor 28 during transport and storage of the single-use cartridge 22 prior to use of the single-use cartridge 22.

The method can comprise taking a measurement of the calibration fluid with the sensor 28 of the single-use cartridge 22 to obtain an actual calibration measurement. The actual calibration measurement can be compared with the known calibration value to determine an offset value corresponding to the difference between the actual calibration measurement and the known calibration value. The offset value can be applied to further measurements made by the specific sensor 28 to calibrate the measurements made by the sensor 28. In an example, if the difference between the actual calibration measurement and the known calibration value exceed the drift limit, the POC system can be configured to provide a notification on the display of the POC system. Additional calibration values for lower and upper value limits, noise limits, or time-to-calibrate limits can be used to establish thresholds for sensor and system performance quality. The POC system can be configured to provide notification on the display of the POC system corresponding to the additional calibration values for lower and upper value limits, noise limits, or time-to-calibrate limits.

TABLE 1
Representative known calibration values and actual calibration
value tolerances
Calibration Specifications

| | | High | | Noise | | Drift | | Time |
|---|---|---|---|---|---|---|---|---|
| Sensor | Low Limit | Limit | | Limit | | Limit | | Given |
| O₂ | BP Dependent | 140 | nA | 0.2 | nA | 0.027 | nA | 180 sec |
| CO₂ | −150 mV | 140 | mV | 0.2 | mV | 0.027 | mV | 180 sec |
| pH | −150 mV | 140 | mV | 0.2 | mV | 0.027 | mV | 180 sec |

| Projection | | | |
|---|---|---|---|
| Sensor | Drift Start Limit | Data points Needed | Good Fit |
| O₂ | 0.10 nA | 10 | 0.95 |

The method can comprise loading the testing chamber 26 with a first LQC fluid. The first LQC fluid can displace calibration fluid pre-loaded into the testing chamber 26 and force the calibration fluid from the testing chamber 26 and into the waste chamber 32. The first LQC fluid can have a first known concentration of the at least one analyte. The first known concentration can correspond to a lower bound of an expected measurement range for the analyte in a biological sample; an upper bound of the expected measurement range; or a midpoint within the expected measurement range. In an example, the first LQC fluid can include more than one analyte each with a first known concentration.

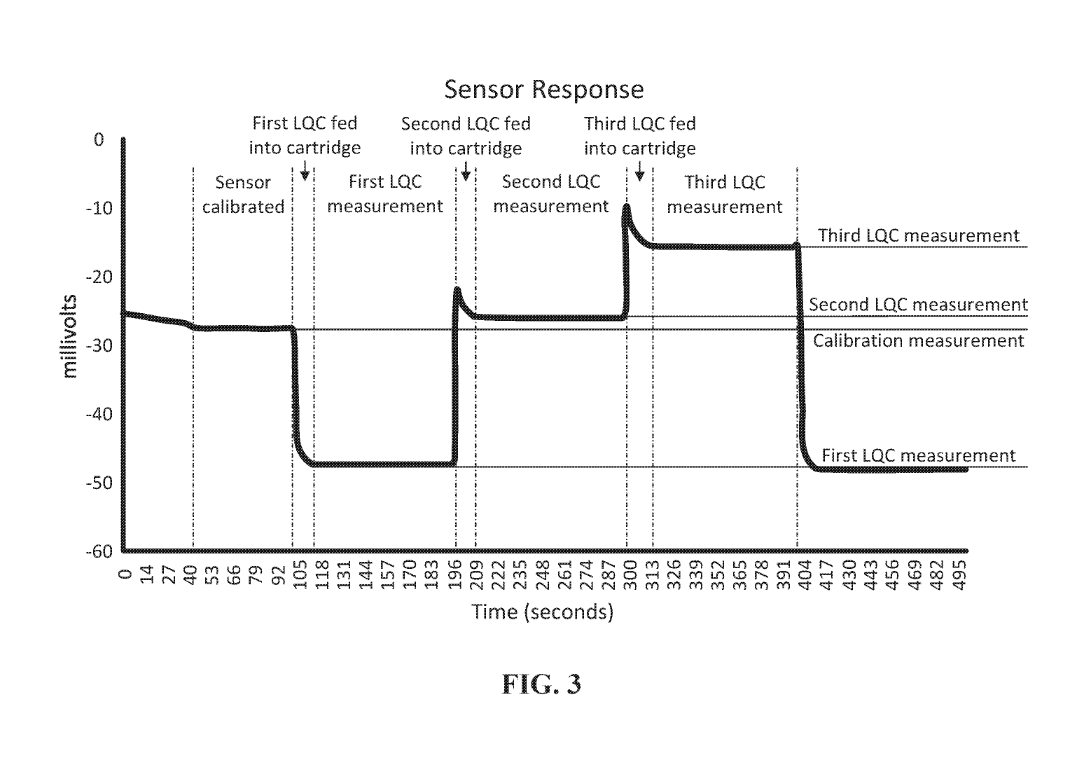

The method can comprise taking a measurement of the first LQC fluid with the sensor 28 of the single-use cartridge 22 to obtain an actual first measurement of the analyte in the first LQC fluid as illustrated in FIG. 3. In at least one example, the offset value can be applied to the measured concentration. The first actual measurement can be compared with the first known concentration to determine a first difference value between the first actual measurement and the first known concentration. The POC system can be configured to display the first measurement value and the first difference value for record taking purposes. The POC system can be configured to provide a notification on the display of the POC system if the first difference value exceeds a predetermined threshold.

The method can comprise loading the testing chamber 26 with a second LQC fluid. The second LQC fluid can displace the first LQC fluid from the testing chamber 26 and into the waste chamber 32. The second LQC fluid can have a second known concentration of the at least one analyte. The second known concentration can correspond to a lower bound of an expected measurement range for the analyte in a biological sample; an upper bound of the expected measurement range; or a midpoint within the expected measurement range. The second LQC fluid can have a second known concentration that differs from the first known concentration of the first LQC fluid such that the second known concentration corresponds to a different portion of the expected measurement range. In an example, the first LQC fluid can include more than one analyte each with a first known concentration.

The method can comprise taking a measurement of the second LQC fluid with the sensor 28 of the single-use cartridge 22 to obtain an actual second measurement of the analyte in the second LQC fluid as illustrated in FIG. 3. In at least one example, the offset value can be applied to the measured concentration. The second actual measurement can be compared with the second known concentration to determine a second difference value between the second actual measurement and the second known concentration. The POC system can be configured to display the second measurement value and the second difference value for record taking purposes. The POC system can be configured to provide a notification on the display of the POC system if the second difference value exceeds a predetermined threshold.

In an example, the method illustrated in FIG. 2A or the method illustrated in FIG. 2B can further comprise loading the testing chamber 26 with a third LQC fluid. The third LQC fluid can displace the second LQC fluid from the testing chamber 26 and into the waste chamber 32. The third LQC fluid can have a second known concentration of the at least one analyte. The third known concentration can correspond to a lower bound of an expected measurement range for the analyte in a biological sample; an upper bound of the expected measurement range; or a midpoint within the expected measurement range. The third LQC fluid can have a third known concentration that differs from the first and second known concentrations of the first and second LQC fluids such that the third known concentration corresponds to a different portion of the expected measurement range. In an example, the third LQC fluid can include more than one analyte each with a first known concentration.

The method can comprise taking a measurement of the third LQC fluid with the sensor 28 of the single-use cartridge 22 to obtain an actual third measurement of the analyte in the third LQC fluid as illustrated in FIG. 3. In at least one example, the offset value can be applied to the measured concentration. The third actual measurement can be compared with the third known concentration to determine a third difference value between the third actual measurement and the third known concentration. The POC system can be configured to display the third measurement value and the third difference value for record taking purposes. The POC system can be configured to provide a notification on the display of the POC system if the third difference value exceeds a predetermined threshold.

The method can comprise taking the measurements of fourth, fifth, and additional LQC fluids can be performed during linearity testing where at least five different analyte levels are evaluated.

In an example, the method illustrated in FIG. 2A or the method illustrated in FIG. 2B can further comprise loading the testing chamber 26 with a biological sample. The biological sample can displace the calibration fluid, first LQC fluid, and the second LQC fluid from the testing chamber 26 and into the waste chamber 32. The sensor 28 can measure the concentration of the at least one analyte within the biological sample. In at least one example, the offset value can be applied to the measured concentration.

### VARIOUS NOTES & EXAMPLES

Example 1 is a method of evaluating a point-of-care (“POC”) system having a sensor positioned within a testing chamber of a single-use cartridge for measuring a concentration of at least one analyte in a biological sample, comprising: loading a first liquid quality control (“LQC”) fluid into the testing chamber, the first LQC fluid having a first known concentration of the at least one analyte; measuring the first LQC fluid with the sensor to obtain a first actual measurement; loading a second LQC fluid into the testing chamber to displace the first LQC fluid from the testing chamber, the second LQC fluid having a second known concentration of the at least one analyte; comparing the second actual measurement with the second known concentration.

In Example 2, the subject matter of Example 1 optionally includes comparing the first actual measurement with the first known concentration; and comparing the second actual measurement with the second known concentration.

In Example 3, the subject matter of any one or more of Examples 1-2 optionally include determining a first difference value between the first actual measurement and the first known concentration; determining a second difference value between the second actual measurement and the second known concentration; and providing a notification if at least one of the first and second difference values exceeds a predetermined threshold.

In Example 4, the subject matter of any one or more of Examples 1-3 optionally include wherein the first known concentration corresponds to a lower bound of an expected measurement range for the at least one analyte in the biological sample; wherein the second known concentration corresponds to an upper bound of the expected measurement range for the at least one analyte in the biological sample.

In Example 5, the subject matter of Example 4 optionally includes loading a third LQC fluid into the testing chamber, the third LQC fluid having a third known concentration of the at least one analyte; and measuring the third LQC fluid with the sensor to obtain a third actual measurement.

In Example 6, the subject matter of Example 5 optionally includes wherein the third known concentration corresponds to a midpoint amount within the expected measurement range for the at least one analyte in the biological sample.

In Example 7, the subject matter of any one or more of Examples 5-6 optionally include loading a fourth LQC fluid into the testing chamber, the fourth LQC fluid having a fourth known concentration of the at least one analyte; measuring the fourth LQC fluid with the sensor to obtain a fourth actual measurement; loading a fifth LQC fluid into the testing chamber, the fifth LQC fluid having a fifth known concentration of the at least one analyte; and measuring the fifth LQC fluid with the sensor to obtain a fifth actual measurement.

In Example 8, the subject matter of Example 7 optionally includes plotting the first, second, third, fourth, and fifth actual measurements; plotting a function intersecting the first, second, third, fourth, and fifth actual measurements; evaluating the linearity of the function.

In Example 9, the subject matter of any one or more of Examples 1-8 optionally include wherein the second LQC fluid is fed into a flow path intersecting the testing chamber at an upstream position.

In Example 10, the subject matter of Example 9 optionally includes wherein a waste receptacle is positioned on the flow path downstream of the testing chamber to receive fluids from the testing chamber.

In Example 11, the subject matter of any one or more of Examples 1-10 optionally include wherein the testing chamber and the sensor are positioned on a single use cartridge operably connectable to a POC system having a display for presenting measurement information collected by the sensor.

In Example 12, the subject matter of any one or more of Examples 1-11 optionally include filling the testing chamber with a calibration fluid before introduction of other fluids, the calibration fluid having a known calibration value; and measuring the calibration fluid with the sensor to obtain an actual calibration measurement.

In Example 13, the subject matter of Example 12 optionally includes comparing the known calibration value to the actual calibration measurement to determine a sensor offset.

In Example 14, the subject matter of Example 13 optionally includes correcting the first actual measurement and the second actual measurement according to the determined sensor offset.

In Example 15, the subject matter of any one or more of Examples 13-14 optionally include loading a biological sample into the testing chamber to displace at least one of the first LQC fluid or the second LQC fluid from the testing chamber; measuring a concentration of the at least one analyte in the biological sample.

In Example 16, the subject matter of Example 15 optionally includes correcting the measured concentration of the at least one analyte in the biological sample according to the determined sensor offset.

In Example 17, the subject matter of Example 16 optionally includes wherein the at least one analyte comprises a gas entrained within the fluid portion.

Example 18 is a POC system for measuring a concentration of at least one analyte in a biological sample, comprising: a POC system having a display; and a single use cartridge defining a flow path intersecting to a testing chamber, the single use cartridge having a sensor positioned within the testing chamber; wherein the single-use cartridge is configured to receive a first LQC fluid into the testing chamber and subsequently receive a second LQC fluid into the testing chamber to displace the first LQC fluid.

In Example 19, the subject matter of Example 18 optionally includes wherein the single use cartridge further comprises a feed port fluidly connected to the flow path upstream of the testing chamber for receiving fluids into the testing chamber.

In Example 20, the subject matter of any one or more of Examples 18-19 optionally include wherein the single use cartridge defines a waste chamber fluidly connected to the flow path downstream of the testing chamber to receive fluids displaced from the testing chamber.

In Example 21, the subject matter of any one or more of Examples 18-20 optionally include wherein the single use cartridge further comprises an interface operably connected to a corresponding interface to transmit sensor information from the sensor to the system circuitry and ultimately to the display.

In Example 22, the subject matter of any one or more of Examples 18-21 optionally include wherein a calibration fluid is initially received within the testing chamber to cover the sensor; wherein introducing the first LQC displaces the calibration fluid from the testing chamber.

In Example 23, the subject matter of any one or more of Examples 18-22 optionally include a display.

In Example 24, the subject matter of any one or more of Examples 18-23 optionally include a communication system for providing information to alternate data sinks.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of“at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

What is claimed is:

1. A method of evaluating a point-of-care (“POC”) system having a sensor positioned within a testing chamber of a single-use cartridge for measuring a concentration of at least one analyte in a biological sample, comprising:
loading a first liquid quality control (“LQC”) fluid into the testing chamber, the first LQC fluid having a first known concentration of the at least one analyte;
measuring the first LQC fluid with the sensor to obtain a first actual measurement;
loading a second LQC fluid into the testing chamber to displace the first LQC fluid from the testing chamber, the second LQC fluid having a second known concentration of the at least one analyte;
comparing the second actual measurement with the second known concentration.

2. The method of claim 1, further comprising:
comparing the first actual measurement with the first known concentration; and
comparing the second actual measurement with the second known concentration.

3. The method of claim 1, further comprising:
determining a first difference value between the first actual measurement and the first known concentration;
determining a second difference value between the second actual measurement and the second known concentration; and
providing a notification if at least one of the first and second difference values exceeds a predetermined threshold.

4. The method of claim 1, wherein the first known concentration corresponds to a lower bound of an expected measurement range for the at least one analyte in the biological sample;
wherein the second known concentration corresponds to an upper bound of the expected measurement range for the at least one analyte in the biological sample.

5. The method of claim 4, further comprising:
loading a third LQC fluid into the testing chamber, the third LQC fluid having a third known concentration of the at least one analyte; and
measuring the third LQC fluid with the sensor to obtain a third actual measurement.

6. The method of claim 5, wherein the third known concentration corresponds to a midpoint amount within the expected measurement range for the at least one analyte in the biological sample.

7. The method of claim 5, further comprising:
loading a fourth LQC fluid into the testing chamber, the fourth LQC fluid having a fourth known concentration of the at least one analyte;
measuring the fourth LQC fluid with the sensor to obtain a fourth actual measurement;
loading a fifth LQC fluid into the testing chamber, the fifth LQC fluid having a fifth known concentration of the at least one analyte; and
measuring the fifth LQC fluid with the sensor to obtain a fifth actual measurement.

8. The method of claim 7, further comprising:
plotting the first, second, third, fourth, and fifth actual measurements;
plotting a function intersecting the first, second, third, fourth, and fifth actual measurements;
evaluating the linearity of the function.

9. The method of claim 1, wherein the second LQC fluid is fed into a flow path intersecting the testing chamber at an upstream position.

10. The method of claim 9, wherein a waste receptacle is positioned on the flow path downstream of the testing chamber to receive fluids from the testing chamber.

11. The method of claim 1, wherein the testing chamber and the sensor are positioned on a single use cartridge operably connectable to a POC system having a display for presenting measurement information collected by the sensor.

12. The method of claim 1, further comprising:
filling the testing chamber with a calibration fluid before introduction of other fluids, the calibration fluid having a known calibration value; and
measuring the calibration fluid with the sensor to obtain an actual calibration measurement.

13. The method of claim 12, further comprising:
comparing the known calibration value to the actual calibration measurement to determine a sensor offset.

14. The method of claim 13, further comprising:
correcting the first actual measurement and the second actual measurement according to the determined sensor offset.

15. The method of claim 13, further comprising:
loading a biological sample into the testing chamber to displace at least one of the first LQC fluid or the second LQC fluid from the testing chamber;
measuring a concentration of the at least one analyte in the biological sample.

16. The method of claim 15, further comprising:
correcting the measured concentration of the at least one analyte in the biological sample according to the determined sensor offset.

17. The method of claim 16, wherein the at least one analyte comprises a gas entrained within the fluid portion.

18. A POC system for measuring a concentration of at least one analyte in a biological sample, comprising:
a POC system having a display; and
a single use cartridge defining a flow path intersecting to a testing chamber, the single use cartridge having a sensor positioned within the testing chamber;
wherein the single-use cartridge is configured to receive a first LQC fluid into the testing chamber and subsequently receive a second LQC fluid into the testing chamber to displace the first LQC fluid.

19. The POC system of claim 18, wherein the single use cartridge further comprises a feed port fluidly connected to the flow path upstream of the testing chamber for receiving fluids into the testing chamber.

20. The POC system of claim 18, wherein the single use cartridge defines a waste chamber fluidly connected to the flow path downstream of the testing chamber to receive fluids displaced from the testing chamber.

21. The POC system of claim 18, wherein the single use cartridge further comprises an interface operably connected to a corresponding interface to transmit sensor information from the sensor to the system circuitry and ultimately to the display.

22. The POC system of claim 18, wherein a calibration fluid is initially received within the testing chamber to cover the sensor;
wherein introducing the first LQC displaces the calibration fluid from the testing chamber.

23. The POC system of claim 18, further comprises a display.

24. The POC system of claim 18, further comprises a communication system for providing information to alternate data sinks.
